(19) United States
(12) Patent Application Publication
(10) Pub. No.: US 2023/0381085 A1
(43) Pub. Date: Nov. 30, 2023

(54) ADHESIVE MASCARA COMPOSITIONS

(71) Applicant: L'ORÉAL, Paris (FR)

(72) Inventor: Sylvie PORET-FRISTOT, Clark, NJ (US)

(73) Assignee: L'ORÉAL, Paris (FR)

(21) Appl. No.: 17/826,413

(22) Filed: May 27, 2022

(51) Int. Cl. A61K 8/60; A61K 8/81; A61Q 1/10

(52) U.S. Cl. CPC A61K 8/60; A61K 8/8111; A61K 8/8147; A61K 8/8152; A61Q 1/10; A61K 2800/43; A61K 2800/48

(57) ABSTRACT

Compositions for providing desired adhesion and/or long-wear, an adhesive mascara may be provided. The adhesive mascara composition may include water, at least about 20% of liquid polybutene, a wax and/or derivative of an acid or alcohol in the C26-C30 range (e.g., having 26-30 carbons), an acrylic polymer (such as Styrene/Acrylates/Ammonium Methacrylate Copolymer and/or Polyacrylate-21 and is free of a latex polymer (such as Acrylates/Ethylhexyl Acrylate Copolymer) having a low glass transition temperature (T_{g}), and optionally a saccharide adhesive (such as caramel). The composition may include one or more pigments. The composition may include a plurality of surfactants, such as a plurality of non-ionic surfactants, and/or an anionic surfactant. The composition may include C2-C8 polyols, a C2-C8 monoalcohol, a cellulosic polymer, a film forming polymer, a thickening agent, a butter, or a combination thereof.

### TECHNICAL FIELD

The present disclosure is drawn to mascara compositions, and adhesive mascara compositions suitable for adhering lash extensions for extended periods of time.

### BACKGROUND

To achieve a desired aesthetic for a user's eyelashes, mascara may be utilized. These mascaras must ideally provide a high degree of smudge resistance, as well as relatively long wear. In some instances, lash extensions may be adhered to a user's lashes using a glue adhesive (e.g., low-viscosity/highly flowable formulations that may have short play times, and are free of waxes). However, some users may wish for the look of lash extensions, but for various reasons, conventional mascaras and adhesives cannot provide the desired look and/or the long wear required.

### BRIEF SUMMARY

To achieve that desired look and/or long-wear, an adhesive mascara may be provided. The adhesive mascara composition may include water, at least about 20% of liquid polybutene, a wax and/or derivative of an acid or alcohol in the C26-C30 range (e.g., having 26-30 carbons), an acrylic polymer (such as Styrene/Acrylates/Ammonium Methacrylate Copolymer and/or Polyacrylate-21 and is free of a latex polymer (such as Acrylates/Ethylhexyl Acrylate Copolymer) having a low glass transition temperature (T_{g}), and optionally a saccharide adhesive (such as caramel).

In some embodiments, the sum of the amount of the acrylic polymer and the amount of the saccharide adhesive may be less than 8% by weight of the composition. In some embodiments, the liquid polybutene may be present in an amount of 20%-25% by weight of the composition. In some embodiments, the acrylic polymer may be present in an amount of 1.5-4% by weight of the composition. In some embodiments, the acrylic polymer may be a styrene acrylate copolymer. In some embodiments, the acrylic polymer may be Styrene/Acrylates/Ammonium Methacrylate Copolymer, Polyacrylate-21, or a combination thereof. In some embodiments, the composition may include one or more waxes and/or one or more pigments. In some embodiments, the composition may include a plurality of surfactants. In some embodiments, the plurality of surfactants may include a plurality of non-ionic surfactants, and/or an anionic surfactant.

In some embodiments, the composition may include a C2-C8 polyol, a C2-C8 monoalcohol, a cellulosic polymer, a film forming polymer, a thickening agent, a butter, or a combination thereof.

In some embodiments, the composition is configured such that a ratio R1 of the amount of the acrylic polymer by weight percent to the amount of the saccharide adhesive by weight percent may be R1>0.3, such as 0.3<R1<2.

In some embodiments, a method is provided. The method may include applying an embodiment of a mascara adhesive composition as disclosed herein to a plurality of eyelashes and applying a lash strip onto the mascara adhesive composition, onto the plurality of eyelashes.

### DETAILED DESCRIPTION

Other than in the operating examples, or where otherwise indicated, all numbers expressing quantities of ingredients and/or reaction conditions are to be understood as being modified in all instances by the term “about” which can encompass ±10%, ±8%, ±6%, ±5%, ±4%, ±3%, ±2%, ±1%, or ±0.5%.

As used herein, the term “at least one” means one or more and thus includes individual components as well as mixtures/combinations.

As used herein, the term “free” or “completely free (of a component)” refers to compositions that do not contain the component in any measurable degree by standard means. As used herein, the term “substantially free (of a component)” refers to compositions that contain no appreciable amount of the component, for example, no more than about 1% by weight, or no more than about 0.5% by weight, or no more than about 0.3% by weight, such as no more than about 0.1% by weight, based on the weight of the composition.

In some embodiments, a mascara adhesive composition may be provided, the composition comprising a plurality of materials. The composition may be an emulsion.

Water

The mascara adhesive composition may include water. In some embodiments, water may be present in amounts of up to 65% by weight of the composition. In some embodiments, water may be present in amounts from 15%, 20%, 25%, or 30% up to 35%, 40%, 45%, 50%, 55%, 60%, or 65% by weight of the composition, including any combinations or subrange thereof. In some embodiments, the water is present in an amount of less than 40% by weight of the composition. In some embodiments, the water is present in an amount of less than 25%-35% by weight of the composition. In some embodiments, the water is present in an amount of less than 30%-35% by weight of the composition.

Acrylic Polymer

In some embodiments, the mascara adhesive composition may include an acrylic polymer having a high glass transition temperature (T_{g}). Such acrylic polymers will have a T_{g} value no less than 0° C., and preferably no less than 10° C., and preferably less than 50° C.

In some embodiments, the acrylic polymer may be a random styrene acrylate copolymer. Non-limiting examples of such acrylic polymers include styrene/acrylates/ammonium methacrylate copolymer or styrene acrylic copolymer. Exemplary commercial random styrene acrylate copolymer products that may be used include, but are not limited to, SYNTRAN 5760 (with or without paraben), by Interpolymer Corporation; JONCRYL 77, by BASF Performance Chemicals; and RHOPLEX P376, by Dow Chemical Company.

In some embodiments, the acrylic polymer may be polyacrylate-21.

In some embodiments, the acrylic polymer comprises or consists of a single polymer. In some embodiments, the acrylic polymer comprises a plurality of polymers. In some embodiments, the acrylic polymer comprises or consists of styrene/acrylates/ammonium methacrylate copolymer, polyacrylate-21, or a combination thereof.

In some embodiments, the acrylic polymer may be present in an amount of 1.5-4% by weight of the composition. In some embodiments, the acrylic polymer may be present in an amount from 1.5%, 2%, 2.5%, 3%, or 3.5% up to 2%, 2.5%, 3%, 3.5%, or 4% by weight of the composition, including any combination or subrange thereof

Liquid Polybutene

The composition may include liquid polybutene. In some embodiments, the composition may be free, or substantially free, of liquid polybutene. In some embodiments, the liquid polybutene may be present in an amount of at least about 20% by weight of the composition. In some embodiments, the liquid polybutene may be present in an amount of at least 20% by weight of the composition. In some embodiments, the amount of liquid polybutene in the composition may be at least 20% and less than 21%, 22%, 23%, 24%, or 25% by weight of the composition.

In some embodiments, the amount of liquid polybutene (by weight percent) relative to the amount of acrylic polymer (by weight percent) is controlled. In some embodiments, the ratio R1, of the amount of the latex polymer by weight percent to the amount of the acrylic polymer by weight percent may be 4<R1<15. In some embodiments, R1 may be 5<R1<10.5.

Saccharide Adhesive

In some embodiments, the mascara adhesive composition may include a saccharide adhesive. As used herein, the term “saccharide adhesive” refers to any adhesive comprising a saccharide. In some embodiments, the saccharide adhesive may include fat-emulsified protein dispersed in a concentrated sugar solution. In some embodiments, the saccharide adhesive may be caramel.

In some embodiments, the saccharide adhesive may be present in an amount of 1-5% by weight of the composition. In some embodiments, the saccharide adhesive may be present in an amount from 1%, 1.25%, 1.5%, or 1.75%, up to 2%, 2.5%, 3%, 3.5%, 4%, 4.5%, or 5% by weight of the composition, including any combination or subrange thereof. In some embodiments, the saccharide adhesive is present in an amount of 2-5% by weight of the composition.

In some embodiments, the relative amount of the acrylic polymer (by weight percent) to the amount of the saccharide adhesive (by weight percent) is controlled. In some embodiments, a ratio R2 of the amount of acrylic acid as a percent by weight of the composition to the amount of saccharide adhesive as a percent by weight of the composition is 0.3<R2<2. In some embodiments, R2 may be 0.15<R2<0.35.

In some embodiments, the composition may be free or substantially free of a saccharide adhesive.

Latex Polymer

The mascara adhesive composition may be free of a latex polymer having a low glass transition temperature (T_{g}). Such latex polymers will have a T_{g} value no greater than 10° C., and preferably no greater than 0° C., and preferably greater than −70° C., −65° C., −60° C., −55° C., −50° C., −45° C., or −40° C.

In some embodiments, the latex polymer may be an acrylic latex polymer. In some embodiments, the acrylic latex polymer may be acrylates/ethylhexyl acrylates copolymer, which may be commercially available from Kobo Products, Inc. and Daito Kasei Kogyo Co., Ltd., under the trade names Daitosol 50005J, Daitosol 5500GM, and/or Daitosol 5500GX. This product may be sold in the form of an emulsion that contains water, ethyl methacrylates/N-butyl acrylates/2-methylhexyl acrylates copolymer (aka acrylates/ethylhexyl acrylates copolymer), and Laureth-20. Daitosol 5000SJ is disclosed to have a glass transition temperature of −13° C. Daitosol 5500GM is disclosed to have a glass transition temperature of −65° C.

In some embodiments, the latex polymer may include, but is not limited to, methyl methacrylate/butyl acrylate/ammonium methacrylate/allyl methacrylate (available from Interpolymer); methyl methacrylate/butyl acrylate/allyl methacrylate (available from Interpolymer); styrene/butadiene/acrylic acid (available from Dow); vinyl acetate (available from Rohm & Haas), styrene/acrylic (available from Rohm & Haas), carboxylated styrene/butadiene (available from Rohm & Haas), and vinyl acetate/ethylene (available from Air Products).

Waxes

In some embodiments, the composition may include one or more waxes. In some embodiments, the composition may include a plurality of waxes.

In some embodiments, the composition may include one or more soft waxes. In some embodiments, the composition may include one or more hard waxes. In some embodiments, the composition may include a soft wax and a hard wax.

As used herein, the term “wax” means a lipophilic compound that is solid at room temperature (25° C.), with a solid/liquid reversible change of state, having a melting point of greater than or equal to 30° C., which may be up to 200° C. and in particular up to 120° C.

As used herein, the term “soft wax” refers to waxes which have a melting point of below about 70° C., and preferably, a melting point of below about 60° C. As used herein, the term “hard wax” refers to waxes other than soft waxes—that is, waxes which have a melting point of equal to or greater than about 70° C., and preferably, a melting point of equal to or greater than about 60° C.

Non-limiting examples of soft waxes include paraffin wax, ozokerite, synthetic beeswax, beeswax, candelilla wax, hydrogenated jojoba wax, palm butter, sumac wax, polyglyceryl beeswax, siliconyl beeswax, ceresin wax, orange peel wax, silicone resin wax, alkyl dimethicone wax, Berry wax, koster wax, siliconyl candelilla wax, and montan wax.

Non-limiting examples of hard waxes include carnauba wax, microcrystalline wax, polyethylene wax, hydrogenated castor oil, wax AC 540, hydrogenated castor wax, wax AC 400, rice bran wax, Alcohol polyethylene wax, sunflower seed wax, fischer-tropsch wax, Chinese insect wax, and shellac wax.

In some embodiments, the composition may include waxes in a total amount of no more than 15%, 13%, or 11% by weight. In some embodiments, the waxes may be present in a total amount of 5-15% by weight of the composition. In some embodiments, the waxes may be present in a total amount from 5%, 6%, 7%, 8%, 9%, or 10% up to 11%, 12%, 13%, 14%, or 15% by weight of the composition, including any combination or subrange thereof.

In some embodiments, the composition may include soft waxes in a total amount of no more than 10% by weight of the composition. In some embodiments, the composition may include soft waxes in a total amount from 4%, 5%, 6%, or 7% up to 8%, 9%, or 10% by weight of the composition, including any combination or subrange thereof.

In some embodiments, the composition may include hard waxes in a total amount of no more than 5% by weight of the composition. In some embodiments, the composition may include hard waxes in a total amount from 1%, 2%, or 3%, up to 3.5%, 4%, 4.5%, or 5% by weight of the composition, including any combination or subrange thereof.

In some embodiments, the composition may be free or substantially free of waxes. In some embodiments, the composition may be free or substantially free of a hard wax. In some embodiments, the composition may be free or substantially free of a soft wax.

Derivatives of Acids or Alcohols in the C26-C30 range

In some embodiments, the composition may include a derivative of an acid or alcohol in the C26-C30 range (e.g., having 26-30 carbons in the acid or alcohol chain). In some embodiments, the derivatives may have a linear fatty chain. In some embodiments, the derivatives may have a branched or non-linear fatty chain.

In some embodiments, the composition may include one or more of derivatives of an acid or alcohol in the C26-C30 range in a total amount of no more than 15%, 13%, or 11% by weight. In some embodiments, the derivatives may be present in a total amount of 5-15% by weight of the composition. In some embodiments, the derivatives may be present in a total amount from 5%, 6%, 7%, 8%, 9%, or 10% up to 11%, 12%, 13%, 14%, or 15% by weight of the composition, including any combination or subrange thereof.

In some embodiments, the composition may include either a wax or a derivative of an acid or alcohol in the C26-C30 range. In some embodiments, the composition may be free or substantially free of a derivative of an acid or alcohol in the C26-C30 range.

Other Materials

In some embodiments, the composition may comprise other materials. The other materials may be present in a total amount of less than 40% by weight of the composition. In some embodiments, the other materials may be present in a total amount of 25-35% by weight of the composition.

In some embodiments, the other materials may comprise or consist of colorants, C2-C8 monoalcohols, C2-C8 polyols, antioxidants, butters, chelating agents, thickening agents, fatty esters, film forming agents, preservatives, and surfactants.

In some embodiments, the compositions may be free or substantially free of silicas and/or clays. As used herein, “clay” refers to silicates containing a cation that may be chosen from calcium, magnesium, aluminum, sodium, potassium, and lithium cations, and mixtures thereof. Non-limiting examples of clays include the smectite family such as montmorillonites, hectorites, bentonites, beidellites, and saponites, and also of the family of vermiculites, stevensite, and chlorites.

Colorants

The compositions of the present invention may optionally further comprise at least one colorant. Suitable colorants (coloring agents) include any colorant typically found in lip compositions. Suitable colorants include, but are not limited to, lipophilic dyes, pigments and pearlescent agents, and their mixtures.

Suitable examples of fat-soluble dyes are, for example, Sudan red, DC Red 17, DC Green 6, β-carotene, soybean oil, Sudan brown, DC Yellow 11, DC Violet 2, DC Orange 5 and quinoline yellow.

Suitable pigments can be white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C Red No. 10, 11, 12, and 13, D&C Red No. 7, D&C Red No. 5 and 6, and D&D Red No. 34, as well as lakes such as D&C Yellow Lake No. 5 and D&C Red Lake No. 2.

Suitable pearlescent pigments can be chosen from, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

The colorant may be present in an amount sufficient to provide color to lashes. In some embodiments, the colorant may be present in a total amount from 0.1% to 20% by weight of the composition. In some embodiments, the colorant may be present in a total amount from 0.1%, 0.5%, 1%, 2%, 3%, 4%, or 5%, up to 10%, 12.5%, 15%, 17.5%, or 20% by weight of the composition, including any combination or subrange thereof. In some embodiments, the colorant may be present in a total amount from 5% to 15% by weight of the composition.

In some embodiments, the colorant may comprise or consist of one or more pigments. In some embodiments, a pigment may be present in a total amount from 0.1% to 20% by weight of the composition. In some embodiments, the pigment may be present in a total amount from 0.1%, 0.5%, 1%, 2%, 3%, 4%, or 5%, up to 10%, 12.5%, 15%, 17.5%, or 20% by weight of the composition, including any combination or subrange thereof. In some embodiments, the pigment may be present in a total amount from 5% to 15% by weight of the composition.

In some embodiments, the composition may be free or substantially free of a colorant. In some embodiments, the composition may be free or substantially free of a pigment.

C2-C8 Monoalcohols

In some embodiments, the composition may include one or more C2-C8 monoalcohols. In some embodiments, a single C2-C8 monoalcohol is utilized. In some embodiments, the C2-C8 monoalcohol is a C2-C8 monoalkanol.

Non-limiting examples of C2-C8 monoalcohols include ethanol and isopropanol. If the C2-C8 monoalcohol is ethanol, the C2-C8 monoalcohol includes any denaturant used to denature the ethanol. In some embodiments, the C2-C8 monoalcohol is a denatured alcohol.

In some embodiments, the C2-C8 monoalcohol may be present in a total amount of 0.1%-5% by weight of the composition. In some embodiments, the C2-C8 monoalcohol may be present in a total amount from 0.1%, 1.5%, 2%, or 3% up to 4%, 4.5%, or 5% by weight of the composition, including any combination or subrange thereof.

In some embodiments, the composition may be free or substantially free of a C2-C8 monoalcohol.

C2-C8 Polyols

In some embodiments, the composition may include one or more C2-C8 polyols. In some embodiments, the composition includes a plurality of C2-C8 polyols.

Non-limiting examples of C2-C8 polyols include glycerol or diols, such as caprylyl glycol, 1,2-pentanediol, propanediol, butanediol, glycols and glycol ethers, such as ethylene glycol, propylene glycol, butylene glycol, dipropylene glycol or diethylene glycol.

In some embodiments, the C2-C8 polyol may be present in a total amount of 0.1%-2% by weight of the composition. In some embodiments, the C2-C8 polyol may be present in a total amount from 0.1%, 0.25%, or 0.5% up to 1%, 1.5%, 1.75%, or 2% by weight of the composition, including any combination or subrange thereof.

In some embodiments, the composition may be free or substantially free of a C2-C8 polyol.

Butters

In some embodiments, the composition may include a butter. In some embodiments, the composition includes a single butter. In some embodiments, the composition includes a plurality of butters.

Non-limiting examples of butters include butyrospermum parkii (shea) butter, Theobroma cacao (cocoa) seed butter, almond butter, aloe butter, apricot kernel butter, avocado butter, coconut cream butter, cupuacu butter, dhupu butter, hemp butter, jojoba butter, kokum butter, macadamia nut butter and mango butter.

In some embodiments, the butter may be present in a total amount of 0.1%-10% by weight of the composition. In some embodiments, the butter may be present in a total amount from 0.1%, 1%, 2%, 3%, 4%, or 5% up to 6%, 7%, 8%, 9%, or 10% by weight of the composition, including any combination or subrange thereof. In some embodiments, the butter may be present in a total amount of 5%-10% by weight of the composition.

In some embodiments, the composition may be free or substantially free of a butter.

Thickening Agent

In some embodiments, the composition may include a thickening agent. In some embodiments, the composition includes a single thickening agent. In some embodiments, the composition includes a plurality of thickening agents.

In some embodiments, the thickening agent may be a polymer of natural origin. Non-limiting examples of polymers of natural origin useful as a thickening agent include thickening polymers comprising at least one sugar unit, for instance nonionic guar gums, optionally modified with C1-C6 hydroxyalkyl groups; biopolysaccharide gums of microbial origin, such as scleroglucan gum or xanthan gum; gums derived from plant exudates, such as gum arabic, ghatti gum, karaya gum, gum tragacanth, carrageenan gum, agar gum and carob gum; pectins; alginates; starches; hydroxy(C1-C6)alkylcelluloses and carboxy(C1-C6)alkylcelluloses.

In some embodiments, the thickening agent may be a cellulosic thickener. Non-limiting examples of cellulose thickeners include hydroxyethylcellulose, hydroxymethylcellulose, hydroxypropylcellulose, methylcellulose, ethylhydroxyethylcellulose, carboxymethylcellulose, and a mixture thereof.

In some embodiments, the thickening agent may be present in a total amount of 0.1%40% by weight of the composition. In some embodiments, the thickening agent may be present in a total amount from 0.1%, 0.25%, or 0.5% up to 1%, 2%, 3%, 4%, 5%, 6%, 7%, 8%, 9%, or 10% by weight of the composition, including any combination or subrange thereof. In some embodiments, the thickening agent may be present in a total amount of 0.1%-2% by weight of the composition.

In some embodiments, the composition may be free or substantially free of a thickening agent.

Fatty Ester

In some embodiments, the composition may include a fatty ester. In some embodiments, the fatty esters may be glycerol (also called “glyceryl”) fatty esters, sucrose fatty esters (also known as “sucrose fatty acid esters”), sorbitan fatty ester, fatty alcohol esters, or mixtures thereof. In some embodiments, the fatty ester comprises or consists of a glycerol fatty ester. In some embodiments, the fatty ester comprises or consists of a sucrose fatty ester. In some embodiments, the fatty ester comprises or consists of a sorbitan fatty ester. In some embodiments, the fatty ester comprises or consists of a fatty alcohol ester.

Non-limiting examples of glycerol fatty esters include: glyceryl caprate, glyceryl caprylate, glyceryl oleate, glyceryl linoleate, glyceryl myristate, glyceryl capromyristate, glyceryl stearate, glyceryl hydroxy stearate, glyceryl isostearate, glyceryl ricinoleate, glyceryl dilaurate, glyceryl dioleate, glyceryl distearate, glyceryl mono/dicaprylate, glyceryl mono/dimyristate, glyceryl stearate palmitate, glyceryl tricaprate/caprylate, caprylic/capric diglyceryl succinate, caprylic/capric glycerides, caprylic/capric/isostearic/adipic triglycerides, caprylic/capric/linoleic triglycerides, caprylic/capric triglycerides, caprylic/capric/stearic triglycerides, glyceryl trilaurate/stearate, glyceryl di/tripalmitostearate, glyceryl di/tritristearate, caprylic triglyceride, caprylic/capric/lauric triglycerides, glyceryl triheptanoate, glyceryl trioctanoate, glyceryl trilaurate, glyceryl trioleate, glyceryl tristearate, glyceryltris-12-hydroxystearate, glyceryl triacetyl hydroxystearate, glyceryl triacetyl ricinioleate, glyceryl triisostearate, glyceryl tribehenate, and mixtures thereof. In some embodiments, the glycerol fatty ester may comprise or consist of glyceryl caprylate.

For sucrose fatty esters, the fatty acid in the sucrose fatty acid ester can be any fatty acid, and can contain between 4 and 28 carbon atoms, typically between 8 and 28 carbon atoms, and typically between 8 and 25 carbon atoms, such as between 8 and 18 carbon atoms, such as 8, 9, 10, 11, 12, 13, 14, 15, 16, 17 and 18 carbon atoms. The fatty acid can be synthetic or naturally occurring and include linear and branched fatty acids. The fatty acids include, but are not limited to, myristic acid, palmitic acid, stearic acid, oleic acid, caproic acid, capric acid, myristic acid, decanoic acid and pelargonic acid.

Non-limiting examples of sucrose fatty acid ester includes sucrose monoesters, diesters, triesters and polyesters, and mixtures thereof, and typically contain sucrose monoesters. The sucrose fatty acid esters may include single fatty acid esters and may include homogeneous mixtures of sucrose esters, containing members with different lengths of fatty acid carbon chain and/or members with different degrees of esterification. For example, the sucrose fatty acid esters may include mixtures of monoesters, diesters, triesters, and/or polyesters.

Non-limiting examples of sorbitan esters include sorbitan monostearate, sorbitan tristearate, sorbitan monolaurate, sorbitan monooleate, sorbitan palmitate, and mixtures thereof.

The fatty alcohol esters may include esters of C₆₋₂₂ fatty acids with a monohydric alcohol and/or esters of C₆₋₂₂ fatty alcohols with a monocarboxylic acid. Likewise, fatty alcohol esters may be esters of C₆₋₁₈ fatty acids with a monohydric alcohol and/or esters of C₆₋₁₈ fatty alcohols with a monocarboxylic acid. Non-limiting examples of fatty alcohol esters include butyl isostearate, butyl oleate, butyl octyl oleate, cetyl palmitate, cetyl octanoate, cetyl laurate, cetyl lactate, cetyl isononanoate, cetyl stearate, diisostearyl fumarate, diisostearyl malate, neopentyl glycol dioctanoate, dibutyl sebacate, di-C₁₂₋₁₃ alkyl malate, dicetearyl dimer dilinoleate, dicetyl adipate, diisocetyl adipate, diisononyl adipate, diisopropyl dimerate, triisostearyl trilinoleate, octodecyl stearoyl stearate, hexyl laurate, hexadecyl isostearate, hexydecyl laurate, hexyldecyl octanoate, hexyldecyl oleate, hexyldecyl palmitate, hexyldecyl stearate, isononyl isononanaote, isostearyl isononate, isohexyl neopentanoate, isohexadecyl stearate, isopropyl isostearate, n-propyl myristate, isopropyl myristate, n-propyl palmitate, isopropyl palmitate, hexacosanyl palmitate, lauryl lactate, octacosanyl palmitate, propylene glycol monolaurate, triacontanyl palmitate, dotriacontanyl palmitate, tetratriacontanyl palmitate, hexacosanyl stearate, octacosanyl stearate, triacontanyl stearate, dotriacontanyl stearate, stearyl lactate, stearyl octanoate, stearyl heptanoate, stearyl stearate, tetratriacontanyl stearate, triarachidin, tributyl citrate, triisostearyl citrate, tri-C₁₂₋₁₃-alkyl citrate, tricaprylin, tricaprylyl citrate, tridecyl behenate, trioctyldodecyl citrate, tridecyl cocoate, tridecyl isononanoate, glyceryl monoricinoleate, 2-octyldecyl palmitate, 2-octyldodecyl myristate or lactate, di(2-ethylhexyl)succinate, tocopheryl acetate, and mixtures thereof.

In some embodiments, the fatty ester may be present in a total amount of 0.01%-1% by weight of the composition. In some embodiments, the fatty ester may be present in a total amount from 0.01% or 0.02% up to 0.03%, 0.05%, 0.1%, 0.5%, or 1% by weight of the composition, including any combination or subrange thereof. In some embodiments, the fatty ester may be present in a total amount of 0.01%-0.03% by weight of the composition.

In some embodiments, the composition may be free or substantially free of a fatty ester.

Film Forming Agent

In some embodiments, the composition may include a film forming agent.

Non-limiting examples of film forming agents (e.g., film forming polymers that are compatible with the other ingredients and form a film after application) include polyvinylpyrrolidones (PVP) and vinyl copolymers, e.g., vinyl pyrrolidone (VP)/hexadecane copolymer, PVP/hexadecene copolymer, and VP/eicosene copolymer.

In some embodiments, the film forming agent may include a polysaccharide film forming agent. In some embodiments, the polysaccharide film forming agents may enable the composition to form a pliable, cohesive, and continuous film when applied. Non-limiting examples of a polysaccharide film forming agent include gellan gum, xanthan gum, agar, carrageenan, locust bean gum, acacia senegal gum, guar gum, and konjac mannan gum.

In some embodiments, the film forming agent may be present in a total amount of 0.1%-5% by weight of the composition. In some embodiments, the film forming agent may be present in a total amount from 0.1, 0.5%, or 1%, 2%, or 3% up to 3%, 3.5%, 4%, 4.5%, or 5% by weight of the composition, including any combination or subrange thereof. In some embodiments, the film forming agent may be present in a total amount of 3%-5% by weight of the composition.

In some embodiments, the composition may be free or substantially free of a film forming agent.

Surfactants

In some embodiments, the compositions may include one or more surfactants. In some embodiments, the compositions may include a plurality of surfactants. In some embodiments, the composition may include a plurality of anionic surfactants and a plurality of nonionic surfactants. In some embodiments, the composition is free or substantially free of cationic surfactants.

In some embodiments, the surfactant may be present in a total amount of 1%-15% by weight of the composition. In some embodiments, the surfactant may be present in a total amount from 1, 2%, 3%, 4% or 5% up to 11%, 12%, 13%, 14%, or 15% by weight of the composition, including any combination or subrange thereof. In some embodiments, the surfactant may be present in a total amount of 5%-12% by weight of the composition.

Anionic Surfactant

In some embodiments, the composition may include at least one anionic surfactant.

Non-limiting examples of anionic surfactants include salts, for example, alkali metal salts such as sodium salts, ammonium salts, amine salts, amino alcohol salts and alkaline-earth metal salts, for example magnesium salts, of the following types of compounds: alkyl sulfates, alkyl ether sulfates, acyl isethionates, acyl glycianates, acyl taurates, acyl amino acids, acyl sarcosinates, sulfosuccinates, sulfonates, the alkyl and acyl groups of all these compounds comprising from 6 to 24 carbon atoms (saturated or unsaturated, linear or branched).

In some embodiments, the anionic surfactant may include at least one alkyl ether sulfate. Non-limiting examples of alkyl ether sulfates include lauryl sulfate, laureth sulfate, and salts and mixtures of these. In some embodiments, the surfactant may be sodium laureth sulfate. In some embodiments, the composition may be free or substantially free of an alkyl ether sulfate.

In some embodiments, the anionic surfactant may comprise or consist of at least one phosphate ester. In some embodiments, the anionic surfactant may comprise or consist of a plurality of phosphate esters.

In some embodiments, the phosphate ester surfactants corresponding to the following formula of formula (1):

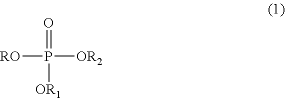

in which R, R₁, and R₂ may independently be hydrogen, an alkyl radical, linear or branched having from 1 to 22 carbon atoms, preferably from 12 to 18 carbon atoms; an oxyalkylenated, linear or branched alkyl radical having from 1 to 22 carbon atoms, and having at least 1, preferably from 2 to 25, more preferably from 2 to 12, moles of ethylene oxide, provided that at least one of the radicals R, R₁, and R₂ is an allyl radical or an oxyalkylene alkyl radical in which the alkyl radical contains at least 6 carbon atoms.

Among the compounds of formula (1), monoesters are preferred in which R₁ and R₂ denote hydrogen and R is chosen from alkyl radicals having 10 to 18 carbon atoms and ethoxylated fatty alcohols having from 10 to 18 atoms. carbon and 2 to 12 moles of ethylene oxide.

Non-limiting examples of compounds of formula (1), include C8-C10 Alkyl Ethyl Phosphate, C9-C15 Alkyl Phosphate, Ceteareth-2 Phosphate, Ceteareth-5 Phosphate, Ceteth-8 Phosphate, Ceteth-10 Phosphate, Cetyl Phosphate, C6-C10 Pareth-4 Phosphate, C12-C15 Pareth-2 Phosphate, C12-C15 Pareth-3 Phosphate, DEA-Ceteareth-2 Phosphate, DEA-Cetyl Phosphate, DEAOleth-3 Phosphate, Potassium cetyl phosphate, Deceth-4 Phosphate, Deceth-6 Phosphate and Trilaureth-4 Phosphate.

In some embodiments, the phosphate ester may be liquid at room temperature (20-25° C.). Non-limiting examples of such phosphate esters include Trilaureth-4 Phosphate, C8-C10 Acid Phosphate, PPG-5-Ceteth-10 Phosphate, Cetoleth-5 Phosphate, Deceth-4 Phosphate, Glycereth-26 Phosphate, Oleth-5 Phosphate, Dioleyl Phosphate, Potassium C12-12 Alkyl Phosphate, TEA C12-13 Alkyl Phosphate, C9-15 Alkyl Phosphate, Oleth-10 Phosphate, DEA Oleth-10 Phosphate, Oleth-3 Phosphate, DEA Oleth-3 Phosphate, Trideceth-10 Phosphate, Trideceth-5 Phosphate, and/or Trideceth-6 Phosphate. In some embodiments, the phosphate ester may be Potassium Cetyl Phosphate, Trideceth-6 Phosphate, or a combination thereof.

In some embodiments, the anionic surfactant may be present in a total amount of 0.1%-5% by weight of the composition. In some embodiments, the anionic surfactant may be present in a total amount from 0.1, 0.5%, or 1%, 1.5%, 2%, or 2.5% up to 3%, 3.5%, 4%, 4.5%, or 5% by weight of the composition, including any combination or subrange thereof. In some embodiments, the anionic surfactant may be present in a total amount of 2%-3% by weight of the composition.

In some embodiments, phosphate ester surfactants may be present in a total amount of 0.1%-5% by weight of the composition. In some embodiments, phosphate ester surfactants may be present in a total amount from 0.1, 0.5%, or 1%, 1.5%, 2%, or 2.5% up to 3%, 3.5%, 4%, 4.5%, or 5% by weight of the composition, including any combination or subrange thereof. In some embodiments, phosphate ester surfactants may be present in a total amount of 2%-3% by weight of the composition.

Nonionic Surfactant

In some embodiments, the composition may include at least one nonionic surfactant. In some embodiments, the composition may include a plurality of nonionic surfactants.

Non-limiting examples of nonionic surfactants include alkoxylated derivatives of the following: fatty alcohols, alkyl phenols, fatty acids, fatty acid esters and fatty acid amides, wherein the alkyl chain is in the C12-050 range, preferably in the C16-C40 range, more preferably in the C24 to C40 range, and having from about 1 to about 110 alkoxy groups. The alkoxy groups may be selected from the group consisting of C2-C6 oxides and their mixtures, with ethylene oxide, propylene oxide, and their mixtures being the preferred alkoxides. The alkyl chain may be linear, branched, saturated, or unsaturated. The alkoxylated alcohols may be used alone or in mixtures thereof.

Other non-limiting examples of ethoxylated fatty alcohols include laureth-3 (a lauryl ethoxylate having an average degree of ethoxylation of 3), laureth-23 (a lauryl ethoxylate having an average degree of ethoxylation of 23), ceteth-10 (a cetyl alcohol ethoxylate having an average degree of ethoxylation of 10), steareth-20 (a stearyl alcohol ethoxylate having an average degree of ethoxylation of 20), steareth-10 (a stearyl alcohol ethoxylate having an average degree of ethoxylation of 10), and steareth-2 (a stearyl alcohol ethoxylate having an average degree of ethoxylation of 2), steareth-100 (a stearyl alcohol ethoxylate having an average degree of ethoxylation of 100), beheneth-5 (a behenyl alcohol ethoxylate having an average degree of ethoxylation of 5), beheneth-10 (a behenyl alcohol ethoxylate having an average degree of ethoxylation of 10), and other derivatives and mixtures of the preceding.

In some embodiments, the nonionic surfactants may include at least one surfactant having a Hydrophilic-Lipophilic Balance (HLB) that is less than 8, and at least one surfactant having a HLB of at least 8. In some embodiments, the nonionic surfactants may comprise or consist of ethoxylated fatty alcohols.

In some embodiments, the nonionic surfactant may be present in a total amount of 1%-10% by weight of the composition. In some embodiments, the nonionic surfactant may be present in a total amount from 1%, 2%, 3%, 4%, or 5% up to 8%, 9%, or 10% by weight of the composition, including any combination or subrange thereof. In some embodiments, the nonionic surfactant may be present in a total amount of 6%-8% by weight of the composition.

Antioxidants, Chelating Agents, and Preservatives

In some embodiments, the composition may include an antioxidant, chelating agent, and/or preservative. In some embodiments, the composition may be substantially free of an antioxidant, a chelating agent, and/or a preservative.

Solvents

In some embodiments, the composition may be free or substantially free of solvents. In some embodiments, the composition may be free of volatile oils. In some embodiments, the composition may be free of one or more volatile hydrocarbon-based oils, silicone oils, or a combination thereof.

As used herein, the term “oil” means a fatty substance that is liquid at room temperature (25° C.) and at atmospheric pressure. The disclosed oils should not fall into one of the other categories of materials described herein. In some embodiment, the at least one oil may include a non-volatile oil, a volatile oil, or a combination thereof.

As used herein, the term “hydrocarbon-based oil” means an oil formed essentially from, or even constituted of, carbon and hydrogen atoms, and optionally oxygen and nitrogen atoms, and not containing any silicon or fluorine atoms. It may contain hydroxy, ester, ether, carboxylic acid, amine and/or amide groups.

As used herein, the term “nonvolatile oil” refers to an oil with a vapor pressure of less than 0.13 Pa.

As used herein, the term “silicone oil” means an oil comprising at least one silicon atom and notably at least one Si—O group.

As used herein, the term “volatile oil” refers to an oil (or non-aqueous medium) that can evaporate on contact with the skin in less than one hour, at room temperature and at atmospheric pressure. The volatile oil is a volatile cosmetic oil, which is liquid at room temperature, notably having a nonzero vapor pressure, at room temperature and at atmospheric pressure, in particular having a vapor pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1300 Pa (0.01 to 10 mmHg).

Volatile Oil

In some embodiments, the volatile oil may be hydrocarbon-based. In some embodiments, the volatile hydrocarbon-based oil may be chosen from hydrocarbon-based oils containing from 7 to 16 carbon atoms.

In some embodiments, the composition may be free or substantially free of one or more volatile branched alkanes. In some embodiments, the composition may be free or substantially free of a volatile hydrocarbon-based oil containing from 7 to 16 carbon atoms may include C8-C16 branched alkanes, such as C8-C16 isoalkanes (also known as isoparaffins), isododecane, isodecane, isohexadecane and for example the oils sold under the trade names Isopar or Permethyl, C8-C16 branched esters such as isohexyl neopentanoate, and mixtures thereof. In some embodiments, the composition may be free or substantially free of a volatile hydrocarbon-based oil containing from 8 to 16 carbon atoms chosen from isododecane, isodecane and isohexadecane, and mixtures thereof, and in particular isododecane.

The composition may be free or substantially free of one or more volatile linear alkanes. In some embodiments, the volatile linear alkane may be liquid at ambient temperature (about 25° C.) and at atmospheric pressure (760 mmHg). In some embodiments, the volatile linear alkane may include a cosmetic linear alkane, which is capable of evaporating on contact with the skin in less than one hour, at ambient temperature (25° C.) and atmospheric pressure (760 mmHg), which is liquid at ambient temperature, in particular having an evaporation rate ranging from 0.01 to 15 mg/cm²/min, at ambient temperature (25° C.) and atmospheric pressure (760 mmHg).

In some embodiments, the composition may be free or substantially free of one or more volatile linear alkanes such as n-heptane (C7), n-octane (C8), n-nonane (C9), n-decane (C10), n-undecane (C11), n-dodecane (C12), n-tridecane (C13), n-tetradecane (C14) and n-pentadecane (C15), and mixtures thereof, and in particular the mixture of n-undecane (C11) and n-tridecane (C13) described in Example 1 of patent application WO 2008/155 059 by the company Cognis. Mention may also be made of n-dodecane (C12) and n-tetradecane (C14) sold by Sasol under the references, respectively, Parafol 12-97 and Parafol 14-97, and also mixtures thereof.

In some embodiments, the composition may be free or substantially free of a volatile silicone oil or solvent that is compatible with cosmetic use.

In some embodiments, the composition may be free or substantially free of volatile silicone oils such as cyclic polysiloxanes and linear polysiloxanes, and mixtures thereof. Volatile linear polysiloxanes that may be mentioned include hexamethyldisiloxane, octamethyltrisiloxane, decamethyltetrasiloxane, tetradecamethylhexasiloxane and hexadecamethylheptasiloxane. Volatile cyclic polysiloxanes that may be mentioned include hexamethylcyclotrisiloxane, octamethylcyclotetrasiloxane, decamethylcyclopentasiloxane and dodecamethylcyclohexasiloxane.

In some embodiments, a method may be provided. The method may be used to provide a desired aesthetic look to an eyelash. The method includes applying a composition according to an embodiment of an adhesive mascara composition as disclosed herein to a plurality of eyelashes, and then applying a lash strip (e.g., lash extensions) onto the composition on the plurality of eyelashes.

### Examples

The following formulas were produced, by combining all oil-soluble components in a first container and heating and mixing until homogenous; adding all remaining components in a second container and heating and mixing until homogenous; combining, cooling, and pouring into an appropriate product container.

TABLE 1
(Exemplary Formulas, Preferred Formulas)

| | Ex 1 | Ex 2 | Ex 3 |
|---|---|---|---|
| Material | (Fla 15) | (Fla 16) | (Fla 11) |
| Water | 30-35% | 30-35% | 30-35% |
| Acrylic Polymer | 3-4% | 1.5-2.5% | 2.5-3.5% |
| soft wax | 6.5-7.5 | 6.5-7.5 | 6.5-7.5 |
| hard wax | 2.5-3.5 | 2.5-3.5 | 2.5-3.5 |
| Polybutene | 20-25% | 20-25% | 20-25% |
| Saccharide Adhesive | — | 4-5% | 2-3% |
| C2-C8 monoalcohol | 2-4% | 2-4% | 2-4% |
| butter | 5-6% | 5-6% | 5-6% |
| C2-C8 polyol | 0.1-1% | 0.1-1% | 0.1-1% |
| colorant (pigment) | 5-10% | 5-10% | 5-10% |
| film former | 0.5-1.5% | 0.5-1.5% | 0.5-1.5% |
| antioxidant | 0.0001-0.5% | 0.0001-0.5% | 0.0001-0.5% |
| chelating agent | 0.0001-0.5% | 0.0001-0.5% | 0.0001-0.5% |
| preservative | 0.5-1% | 0.5-1% | 0.5-1% |
| surfactant (anionic, | 2-3% | 2-3% | 2-3% |
| phosphate ester) | | | |
| surfactant (anionic) | 0.01-1% | 0.01-1% | 0.01-1% |
| surfactant | 5-6% | 5-6% | 5-6% |
| (nonionic, HLB >8) | | | |
| surfactant | 2-3% | 2-3% | 2-3% |
| (nonionic, HLB <8) | | | |
| thickening agent | 3-4% | 3-4% | 3-4% |

TABLE 2
(Comparative Formulas)

| | C1 | C2 | C3 | C4 |
|---|---|---|---|---|
| Material | (Fla 1) | (Fla 5) | (Fla 10) | (Fla 12) |
| Water | 42-48% | 35-40% | 35-40% | 35-40% |
| Acrylic Polymer | 11-12% | 5-6% | 6-7% | 7-8% |
| soft wax | 6.5-7.5 | 6.5-7.5 | 6.5-7.5 | 6.5-7.5 |
| hard wax | 2.5-3.5 | 2.5-3.5 | 2.5-3.5 | 2.5-3.5 |
| Polybutene | — | 10-15% | 10-15% | 3-7% |
| Saccharide Adhesive | — | 1-2% | 3-4% | 4.5-5.5% |
| C2-C8 monoalcohol | 2-4% | 2-4% | 2-4% | 2-4% |
| butter | 5-6% | 5-6% | 5-6% | 5-6% |
| C2-C8 polyol | 0.1-1% | 0.1-1% | 0.1-1% | 0.1-1% |
| colorant (pigment) | 5-10% | 5-10% | 5-10% | 5-10% |
| film former | 0.5-1.5% | 0.5-1.5% | 0.5-1.5% | 0.5-1.5% |
| antioxidant | 0.0001-0.5% | 0.0001-0.5% | 0.0001-0.5% | 0.0001-0.5% |
| chelating agent | 0.0001-0.5% | 0.0001-0.5% | 0.0001-0.5% | 0.0001-0.5% |
| preservative | 0.5-1.5% | 0.5-1% | 0.5-1% | 0.5-1% |
| surfactant (anionic, | 2-3% | 2-3% | 2-3% | 2-3% |
| phosphate ester) | | | | |
| surfactant (anionic) | 0.01-1% | 0.01-1% | 0.01-1% | 0.01-1% |
| surfactant | 5-6% | 5-6% | 5-6% | 5-6% |
| (nonionic, HLB >8) | | | | |
| surfactant | 2-3% | 2-3% | 2-3% | 2-3% |
| (nonionic, HLB <8) | | | | |
| thickening agent | 3-4% | 3-4% | 3-4% | 3-4% |

Each composition was then evaluated for sebum resistance and adhesion, as described below.

Evaluation (Sebum Resistance)

Each composition was applied onto fake lashes and was left to dry at room temperature (25° C.) for 1 hour. The strength of the film obtained was evaluated by immersing the fake lashes in 0.5 ml of sebum for 1 hour. After immersion, the surface of the fake lashes was rubbed with paper, and the state of the film was then observed (degraded or undegraded appearance of the film). The traces on the paper are qualitatively scored from 1 (no/few traces, very good resistance) to 5 (lots of traves, very poor resistance).

As seen in Table 3, below, the comparative formulas with compositions outside the ranges disclosed herein, surprisingly resulted in the composition still having acceptable sebum resistance (e.g., a sebum resistance score ≥3). Here, it is clear that while the reduction of the amount of acrylic polymer and the increase in the amount of polyisobutene can harm sebum resistance, especially in view of the drop in sebum resistance from C1 to C4, it is surprising how little the addition of a large amount of polyisobutene (as seen in Ex 1 and Ex 2) has on sebum resistance.

TABLE 3
(Partial List of Sebum Resistance Evaluation Results)

| | High T_{g} Acrylic | | |
|---|---|---|---|
| | Polymer | Polyisobutene | Sebum Resistance |
| | (% by Wt) | (% by Wt) | (Lower Is Better) |
| Ex 1 (Fla 15) | 3-4% | 20-25% | 3 |
| Ex 2 (Fla 16) | 1.5-2.5% | 20-25% | 3 |
| C 1 (Fla 1) | 11-12% | — | 1 |
| C 4 (Fla 12) | 7-8% | 3-7% | 2 |

Evaluation (Adhesion)

To test adhesion, 20 strokes of each composition was applied to fake eyelashes, and allowed to dry for between 10 minutes and 2 hours. Then, a paper was applied over the eyelashes, and a 500-gram weight was applied for 1 minute. If the paper adheres, the test continues by applying a hair dryer at 60° C. for up to 5 minutes, while a rotative motor rotating at, e.g., 12 rpm, brushes against the paper. The adhesion score is the time (in minutes) at which the paper dropped off of the eyelashes.

As seen in Table 4, below, compositions with lower amounts of acrylic polymer and higher amount of polyisobutene result in greater adhesion.

TABLE 4
(Partial List of Evaluation Results)

| | High Tg Acrylic | | |
|---|---|---|---|
| | Polymer | Polyisobutene | Adhesion |
| | (% by Wt) | (% by Wt) | (Higher Is Better) |
| Ex 1 (Fla 15) | 3-4% | 20-25% | 4.13 |
| Ex 2 (Fla 16) | 1.5-2.5% | 20-25% | 5 |
| Ex 3 (Fla 11) | 2.5-3.5% | 20-25% | 5 |
| C 1 (Fla 1) | 11-12% | — | 1.02 |
| C 2 (Fla 5) | 5-6% | 10-15% | 2 |
| C 3 (Fla 10) | 6-7% | 10-15% | 0.11 |
| C 4 (Fla 12) | 7-8% | 3-7% | 1.8 |

Together, these variables may be adjusted to achieve desired characteristics.

Those skilled in the art will recognize or be able to ascertain using no more than routine experimentation many equivalents to the specific embodiments of the invention described herein. Such equivalents are intended to be encompassed by the following claims.

1-16. (canceled)

17. A method, comprising:
applying a composition to a plurality of eyelashes, the composition comprising:
water;
liquid polybutene in an amount of at least about 20% by weight of the composition of liquid polybutene;
an acrylic polymer having a high Tg; and
a wax and/or derivative of an acids or alcohols having 26-30 carbons;
wherein the composition is free of a latex polymer having a low glass transition temperature (T_{g}); and
applying a lash strip onto the composition on the plurality of eyelashes.

18. The method of claim 17, wherein the composition further comprises a saccharide adhesive.

19. The method of claim 18, wherein the saccharide adhesive is caramel.

20. The method of claim 18, wherein a sum of the amount of the acrylic polymer and the amount of the saccharide adhesive is less than 8% by weight of the composition.

21. The method of claim 18, wherein a ratio R1 of the amount of acrylic acid as a percent by weight of the composition to the amount of saccharide adhesive as a percent by weight of the composition is 0.3<R1<2.

22. The method of claim 17, wherein the liquid polybutene is present in an amount of 20%-25% by weight of the composition.

23. The method of claim 17, wherein the acrylic polymer is present in an amount of 1.5-4% by weight of the composition.

24. The method of claim 17, wherein the water is present in an amount of 25-35% by weight of the composition.

25. The method of claim 17, wherein the acrylic polymer is a styrene acrylate copolymer.

26. The method of claim 17, wherein the acrylic polymer is Styrene/Acrylates/Ammonium Methacrylate Copolymer, Polyacrylate-21, or a combination thereof.

27. The method of claim 17, further comprising one or more waxes.

28. The method of claim 17, further comprising a pigment.

29. The method of claim 17, further comprising a plurality of surfactants.

30. The method of claim 29, wherein the plurality of surfactants comprises a plurality of non-ionic surfactants.

31. The method of claim 29, wherein the plurality of surfactants further comprises an anionic surfactant.

32. The method of claim 17, wherein the composition further comprises a plurality of C2-C8 polyols, a C2-C8 monoalcohol, a cellulosic polymer, a film forming polymer, a thickening agent, a butter, or a combination thereof.
